(19) United States
(12) Patent Application Publication
(10) Pub. No.: US 2025/0345536 A1
(43) Pub. Date: Nov. 13, 2025

(54) Endotracheal tube position anomaly alerting device

(71) Applicant: Kaohsiung Medical University, Kaohsiung (TW)

(72) Inventors: Ming-Ju Tsai, Kaohsiung (TW); Wei-An Chang, Kaohsiung (TW); Chih-Hung Cheng, Kaohsiung (TW); Yeong-Cheng Liou (Pao), Kaohsiung (TW); Chin-Jing Lee, Kaohsiung (TW); Chen-Yi Chou, Kaohsiung (TW); Chien-Chang Wu, Kaohsiung (TW); Deng-chiung Shih, Kaohsiung (TW); Chia-Lin Hsu, Kaohsiung (TW); Li-Li Wang, Chiayi County (TW)

(21) Appl. No.: 18/746,502

(22) Filed: Jun. 18, 2024

(30) Foreign Application Priority Data
May 9, 2024 (TW) 113117248

(51) Int. Cl. A61M 16/00; A61M 16/04; G16H 30/40

(52) U.S. Cl. CPC A61M 16/0051; A61M 16/024; G16H 30/40; A61M 16/0465; A61M 2205/18; A61M 2205/3327; A61M 2205/502; A61M 2205/581; A61M 2205/583; A61M 2210/065

(57) ABSTRACT

An endotracheal tube position anomaly alerting device, for monitoring correctness of the position of an endotracheal tube (ETT), includes: a monitoring module for collecting patients' chest X-ray image data; an object detection module for detecting objects with a deep learning model of YOLO V5; a position evaluation module for reading and determining, with an artificial intelligence (AI) algorithm model, appropriateness of the position of the endotracheal tube according to a detection result from the object detection module; and a display module for displaying an evaluation result about endotracheal tube position appropriateness and sending an alert for clinical reference as needed. The device assists clinical professionals in quickly and accurately determining whether the position of the endotracheal tube is appropriate, so as to reduce unplanned endotracheal tube detachment rate or incidence rate of one lung ventilation (OLV), enhance medical care quality, and enhance patients' safety and stability of vital signs.

### FIELD OF THE INVENTION

The present invention relates to an endotracheal tube position anomaly alerting device. More particularly, the present invention relates to a medical image, especially assisting clinical professionals in quickly and accurately determining whether the position of an endotracheal tube is appropriate, so as to reduce unplanned endotracheal tube detachment rate or incidence rate of one lung ventilation (OLV), enhance medical care quality, and enhance patients' safety and stability of vital signs.

### DESCRIPTION OF THE PRIOR ART

Despite their low incidence rates, unplanned endotracheal tube (ETT) detachment and one lung ventilation (OLV) lead to unstable vital signs of patients, increase the average number of days of a single inpatient hospital stay, incur higher medical cost, and even cause severe harm, such as death, to patients; thus, the correctness of endotracheal tube positions is clinically important. Moreover, every patient staying in an intensive care unit (ICU) has to receive a chest X-ray every two to five days on average. Chest X-rays are nowadays read by physicians who are typically busy and thereby are focused on major lesions, such as pneumonia, while reading the chest X-rays; as a result, sometimes physicians are too busy to check the chest X-rays for the correctness of endotracheal tube positions one by one.

As mentioned above, sometimes unplanned endotracheal tube detachment and one lung ventilation (OLV) occur in ICUs and may be fatal to patients. Theoretically speaking, the position of an endotracheal tube shown in each chest X-ray image must be examined thoroughly. However, nowadays physicians are typically busy and thereby are focused on conspicuous lesions while reading chest X-rays; as a result, sometimes physicians are unaware of incorrect endotracheal tube positions. Therefore, it is necessary to provide a means of detecting and determining the position of an endotracheal tube to thereby assist physicians in determining important reference information about endotracheal tube position appropriateness.

### BRIEF SUMMARY OF THE INVENTION

Therefore, the main purpose of the present invention is to overcome the aforesaid drawbacks of the prior art by providing an endotracheal tube position anomaly alerting device that uses an artificial intelligence (AI) algorithm model for assisting with automatic identification of the position of an endotracheal tube shown in a chest X-ray image to assist clinical professionals in quickly and accurately determining whether the position of the endotracheal tube is appropriate, so as to reduce unplanned endotracheal tube detachment rate or incidence rate of one lung ventilation (OLV), enhance medical care quality, and enhance patients' safety and stability of vital signs.

Another purpose of the present invention is to provide an endotracheal tube position anomaly alerting device that uses an artificial intelligence (AI) algorithm model for assisting with automatically reading and determining the appropriateness of the position of the endotracheal tube shown in a chest X-ray image and sending an appropriate alert, with the artificial intelligence (AI) algorithm model exhibiting satisfactory performance during both the development stage and the testing stage and being applied to clinical practice to enhance patients' care quality and safety.

Yet another purpose of the present invention is to provide an endotracheal tube position anomaly alerting device that evaluates endotracheal tube position appropriateness, on the assumption that every patient staying in an intensive care unit (ICU) has to receive a chest X-ray every two to five days on average, by performing artificial intelligence (AI) algorithm model training with chest X-ray image samples and then using the artificial intelligence (AI) algorithm model to: 1. determine the position of a tracheal carina shown in a chest X-ray image, determine whether an endotracheal tube or tracheostomy tube is in use, and determine its position; 2. calculate the appropriateness of the endotracheal tube position and send an alert.

Still yet another purpose of the present invention is to provide a distance zooming method related to image processing and especially adapted for use in image analysis and marking. Image processing entails calculating and measuring the distance between objects shown in an image to facilitate image analysis and marking. However, directly using pixel values to express a distance is likely to have an inaccurate or confusing outcome, because images differ in size and ratio. Therefore, it is necessary to provide an effective method of converting the pixel values of images into a distance in reality so as to perform image analysis and marking precisely. The method comprises the steps of:
1. defining the original size of a training set image, including the height and width of the image;
2. receiving an input image to be processed;
3. calculating zooming ratio r, wherein r denotes the ratio of the input image size to the training set image size, selecting the least one as the zooming ratio;
4. converting the pixel values of the input image into an actual distance corresponding to the training set image according to the calculated zooming ratio r; and
5. applying the actual distance to image analysis and marking, such as calculating the distance between objects and marking them.

To achieve the above purposes, the present invention is an endotracheal tube position anomaly alerting device for monitoring the correctness of the position of an endotracheal tube, comprising:
a monitoring module for collecting patients' chest X-ray image data;
an object detection module connected to the monitoring module to receive the chest X-ray image data from the monitoring module and use an artificial intelligence (AI) algorithm model to identify the presence of a tracheal carina, an endotracheal tube (ETT) and a tracheostomy tube in the image and mark their positions;
a position evaluation module connected to the object detection module to automatically measure a distance between the endotracheal tube tip and the tracheal carina upon detection of the presence of the tracheal carina and the endotracheal tube by the object detection module and according to the mark positions of a determination result of the artificial intelligence (AI) algorithm model, generate an evaluation result of the endotracheal tube position appropriateness according to the distance thus measured, and trigger an alert when the evaluation result indicates that the position of the endotracheal tube does not fall within a correct range; and
a display module connected to the position evaluation module to receive the determination result of the object detection module and the position evaluation module, send an alerting signal, and display an object marking result and the evaluation result of the endotracheal tube position appropriateness.

In the aforesaid embodiment of the present invention, the object detection module, based on a deep learning model of YOLOv5, inputs a plurality of chest X-ray image data, treats the plurality of chest X-ray image data as a training dataset and a testing dataset for deep learning according to a predetermined ratio, performs model training with the training dataset, performs testing with the testing dataset, performs verification to finish training the artificial intelligence (AI) algorithm model, and evaluates the performance of the artificial intelligence (AI) algorithm model according to a standard performance evaluation indicator.

In the aforesaid embodiment of the present invention, the standard performance evaluation indicator includes but is not limited to precision, recall, mean average precision (mAP@50), and accuracy.

In the aforesaid embodiment of the present invention, the position evaluation module triggers an alert when the endotracheal tube tip is lower than the tracheal carina or when the distance between the endotracheal tube tip and the tracheal carina is less than 3 cm or greater than 5 cm.

In the aforesaid embodiment of the present invention, the chest X-ray image data of the object detection module includes chest X-ray images indicative of the presence of the endotracheal tube, chest X-ray images indicative of the presence of the tracheostomy tube, and chest X-ray images indicative of the absence of the endotracheal tube or tracheostomy tube.

In the aforesaid embodiment of the present invention, the chest X-ray images indicative of the presence of the endotracheal tube mainly originate from the chest X-rays of patients who need to undergo endotracheal tube-based therapy, including cases where the endotracheal tube positions are appropriate and cases where the endotracheal tube positions are inappropriate, with the patients differing in endotracheal tube experience and thoracic anatomical structures.

### BRIEF DESCRIPTION OF THE DRAWINGS

The present invention will be better understood from the following detailed description of preferred embodiments of the invention, taken in conjunction with the accompanying drawings, in which

FIG. 1 is a schematic view of the framework of an endotracheal tube position anomaly alerting device according to the present invention;

FIG. 2 is a schematic view of a process flow of the use of the present invention;

FIG. 3A is a schematic view showing that an artificial intelligence (AI) algorithm model determines that an endotracheal tube is absent according to the present invention;

FIG. 3B is a schematic view showing that the artificial intelligence (AI) algorithm model determines that the position of the endotracheal tube is correct according to the present invention;

FIG. 3C is a schematic view showing that the artificial intelligence (AI) algorithm model determines that the position of the endotracheal tube is abnormal according to the present invention;

FIG. 3D is a schematic view showing that the artificial intelligence (AI) algorithm model determines that the position of the endotracheal tube approximates to an abnormal distance range according to the present invention;

FIG. 4A is a schematic view of an image showing a distance that is the shortest among three distances determined by the artificial intelligence (AI) algorithm model according to the present invention;

FIG. 4B is a schematic view of an image showing that the artificial intelligence (AI) algorithm model detects the position of a tracheal carina but does not detect any endotracheal tube according to the present invention;

FIG. 4C is a schematic view of an image showing that the artificial intelligence (AI) algorithm model detects a tracheal carina and a tracheostomy tube according to the present invention;

FIG. 4D is a schematic view of an image showing that the artificial intelligence (AI) algorithm model detects a tracheal carina and an endotracheal tube and determines the position of the endotracheal tube tip falls within a normal distance range according to the present invention;

FIG. 4E is a schematic view of an image showing that the artificial intelligence (AI) algorithm model detects a tracheal carina and an endotracheal tube and determines that the distance between the endotracheal tube tip and the tracheal carina is less than 3 cm according to the present invention; and

FIG. 4F is a schematic view of an image showing that the artificial intelligence (AI) algorithm model detects a tracheal carina and an endotracheal tube and determines that the distance between the endotracheal tube tip and the tracheal carina is greater than 5 cm according to the present invention.

### DESCRIPTION OF THE PREFERRED EMBODIMENTS

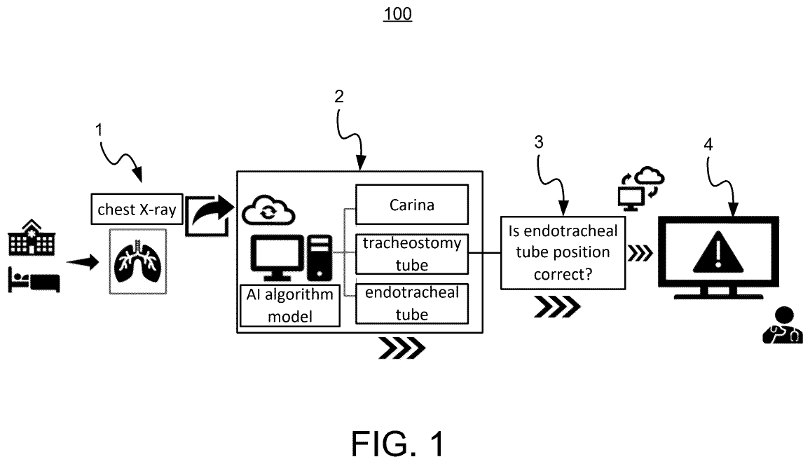

Referring to FIG. 1 through FIG. 4F, there are shown a schematic view of the framework of an endotracheal tube position anomaly alerting device according to the present invention, a schematic view of a process flow of the use of the present invention, a schematic view showing that an artificial intelligence (AI) algorithm model determines that an endotracheal tube is absent according to the present invention, a schematic view showing that the artificial intelligence (AI) algorithm model determines that the position of the endotracheal tube is correct according to the present invention, a schematic view showing that the artificial intelligence (AI) algorithm model determines that the position of the endotracheal tube is abnormal according to the present invention, a schematic view showing that the artificial intelligence (AI) algorithm model determines that the position of the endotracheal tube approximates to an abnormal distance range according to the present invention, a schematic view of an image showing a distance that is the shortest among three distances determined by the artificial intelligence (AI) algorithm model according to the present invention, a schematic view of an image showing that the artificial intelligence (AI) algorithm model detects the position of a tracheal carina but does not detect any endotracheal tube according to the present invention, a schematic view of an image showing that the artificial intelligence (AI) algorithm model detects a tracheal carina and a tracheostomy tube according to the present invention, a schematic view of an image showing that the artificial intelligence (AI) algorithm model detects a tracheal carina and an endotracheal tube and determines the position of the endotracheal tube tip falls within a normal distance range according to the present invention, a schematic view of an image showing that the artificial intelligence (AI) algorithm model detects a tracheal carina and an endotracheal tube and determines that the distance between the endotracheal tube tip and the tracheal carina is less than 3 cm according to the present invention, and a schematic view of an image showing that the artificial intelligence (AI) algorithm model detects a tracheal carina and an endotracheal tube and determines that the distance between the endotracheal tube tip and the tracheal carina is greater than 5 cm according to the present invention. As shown in the diagrams, the present invention is an endotracheal tube position anomaly alerting device that is adapted to monitor the correctness of the position of an endotracheal tube and comprises a monitoring module 1, an object detection module 2, a position evaluation module 3, and a display module 4.

The monitoring module 1 collects patients' chest X-ray image data.

The object detection module 2 is connected to the monitoring module 1 to perform object detection with a deep learning model of YOLO V5.

The position evaluation module 3 is connected to the object detection module 2 to read and determine endotracheal tube position appropriateness according to the detection result of the object detection module 2.

The display module 4 is connected to the position evaluation module 3 to display the evaluation result of the endotracheal tube position appropriateness and send an alert for clinical reference as needed. Therefore, the aforesaid structural features together constitute a novel endotracheal tube position anomaly alerting device 100.

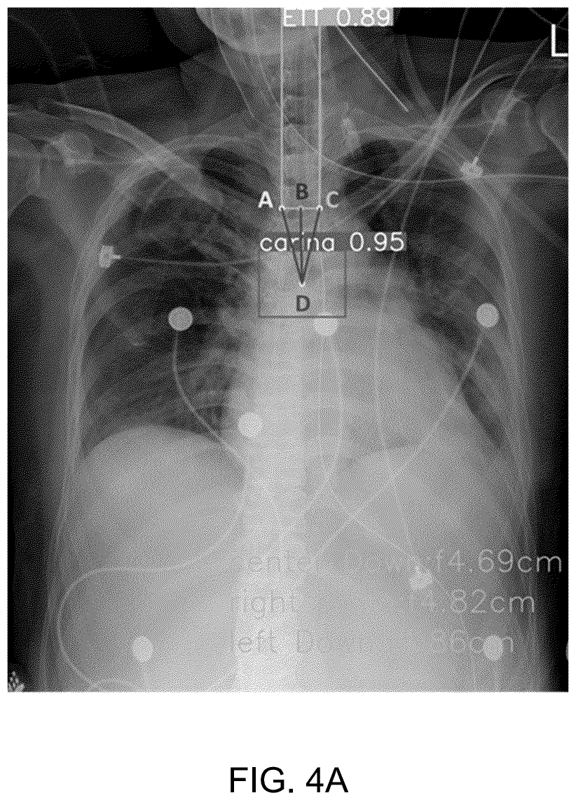

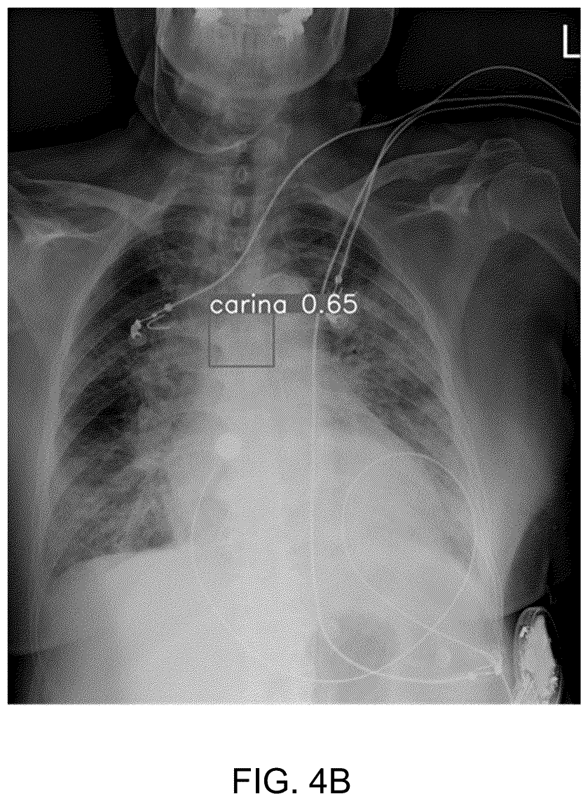

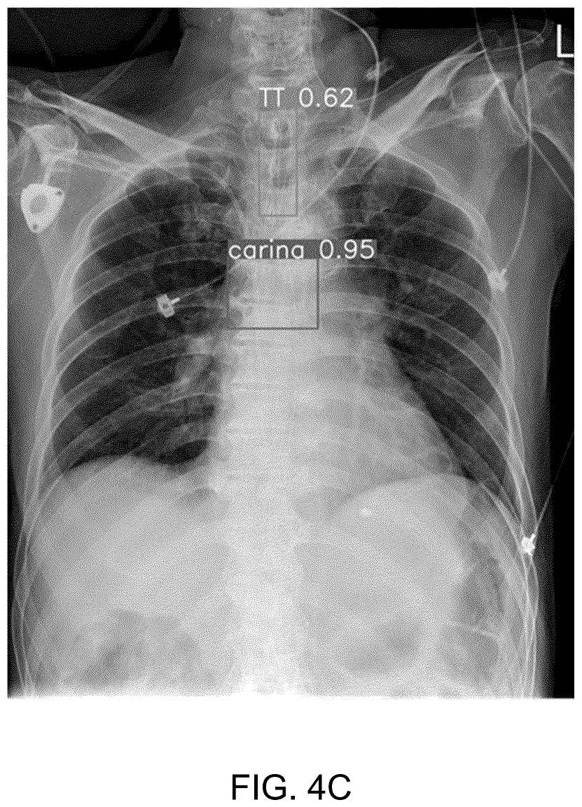

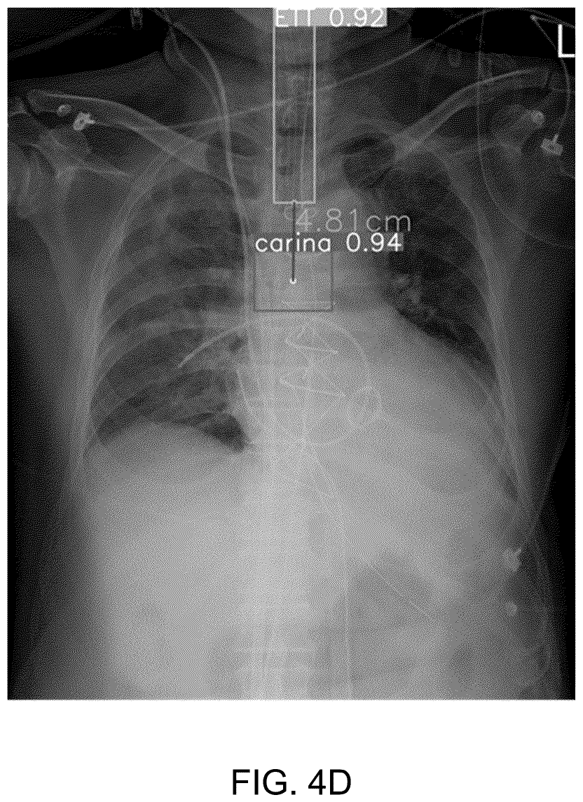

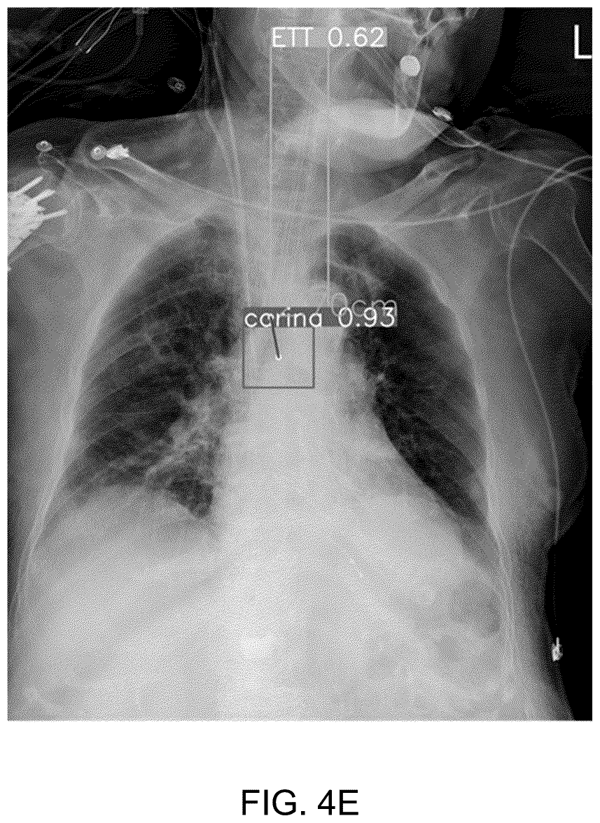

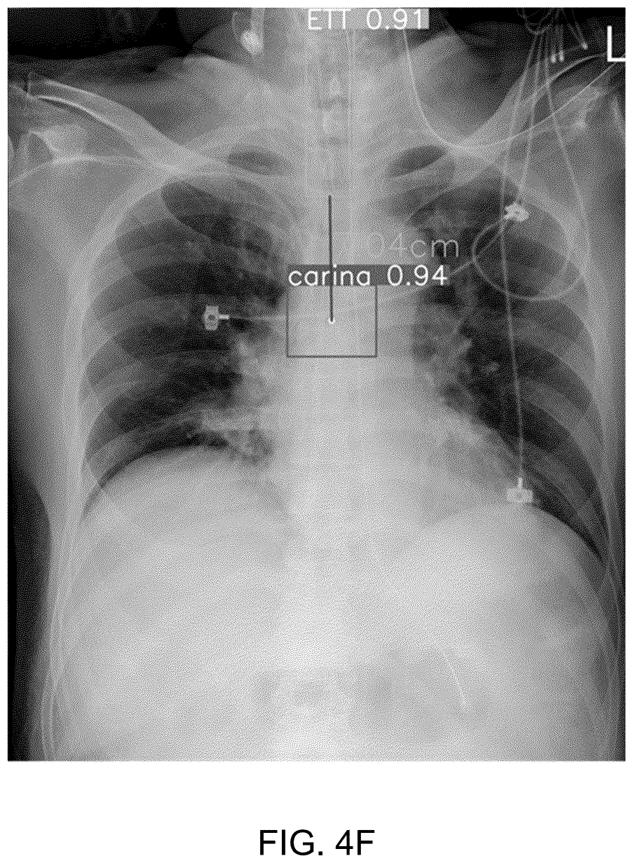

The aforesaid distance measurement process is performed by a position distance appropriateness calculation module and entails undergoing a code check to determine whether it detects at least two objects (ret), determine whether the first object is a tracheal carina, and determine whether the second object is an endotracheal tube, or vice versa. If the tracheal carina and the endotracheal tube are detected, the code will extract corresponding coordinate information from the detected objects. Then, the code calculates the distance between the endotracheal tube and the tracheal carina. The calculation process comprises the steps as follows: 1. calculating the linear distance between two pairs of coordinates with the Euclidean distance equation; 2. performing image prediction with the distance calculated, performing ratio zooming on training set images, and converting pixel values of the distance into the actual distance. The endotracheal tube tip position is calculated in the way as follows: select the leftmost coordinate pair and the rightmost coordinate pair on the lower edge of the marking bounding box of the identified endotracheal tube tip and the midpoint between two points calculated with a function, for example, as shown in FIG. 4A, points A, C are the rightmost and leftmost points at the bottom of the bounding box respectively; point B is the midpoint at the bottom of the bounding box, i.e., the midpoint between points A, C; the distance from a tracheal carina (such as point D in FIG. 4A) is calculated with points A, B, C, and the shortest distance is selected as the result, displaying the related coordinate pairs and distance marks, including a distance and an alert, in the image.

FIGS. 4B˜4F illustrate examples of the results of the determination process carried out by the artificial intelligence (AI) algorithm model of the present invention. FIG. 4B shows that the display model detects the position of the tracheal carina but does not detect any endotracheal tube. FIG. 4C shows that the display model detects the tracheal carina and the tracheostomy tube. FIG. 4D shows that the display model detects the tracheal carina and the endotracheal tube, with the endotracheal tube tip position lying within the normal distance range. FIG. 4E shows that the display model detects the tracheal carina and the endotracheal tube, wherein the distance between the endotracheal tube tip and the tracheal carina is less than 3 cm. FIG. 4F shows that the display model detects the tracheal carina and the endotracheal tube, wherein the distance between the endotracheal tube tip and the tracheal carina is greater than 5 cm.

When it is in use, the present invention is characterized in that the object detection module 2 identifies the presence of the tracheal carina, endotracheal tube (ETT), and tracheostomy tube and mark positions thereof in the chest X-ray image data inputted by the monitoring module 1 with an artificial intelligence (AI) algorithm model. Then, when the object detection module 2 detects the presence of the tracheal carina and the endotracheal tube, the position evaluation module 3 automatically measures the distance between the endotracheal tube tip and the tracheal carina according to the mark positions of the determination result of the artificial intelligence (AI) algorithm model and generates an evaluation result of endotracheal tube position appropriateness according to the distance thus measured. When the evaluation result shows that the position of the endotracheal tube does not fall within the correct range, an alert will be triggered. Finally, in response to the determination results of both the object detection module 2 and the position evaluation module 3, the display module 4 sends an alerting signal as well as displays an object marking result and the evaluation result of endotracheal tube position appropriateness. Therefore, the endotracheal tube position anomaly alerting device 100 can be widely applied to medical-related fields. The present invention puts forth using artificial intelligence (AI) to detect and determine the endotracheal tube position and thus assist physicians in determining important reference information about endotracheal tube position appropriateness, so as to reduce clinical physicians' workload.

### Purpose of Use of the Present Invention:

Both the object detection module 2 and the position evaluation module 3, which are aimed at evaluating endotracheal tube position appropriateness, use the artificial intelligence (AI) algorithm model to evaluate the position of an endotracheal tube shown in a chest X-ray image of a patient. The main purpose of the use of the present invention is to assist clinical professionals in quickly and accurately determining whether the position of an endotracheal tube is appropriate, to reduce unplanned endotracheal tube detachment rate or incidence rate of one lung ventilation (OLV), enhance medical care quality, and enhance patients' safety and stability of vital signs.

The operating principle of the present invention is as follows:

The object detection module 2 performs training with a YOLO V5 model, uses 2278 image samples to perform training, and uses 253 images to perform testing and verification. Predict tracheal carina position in an X-ray image, and confirm whether an endotracheal tube or tracheostomy tube is presented. If an endotracheal tube is detected, the position evaluation module 3 will further calculate the distance between the endotracheal tube tip and the tracheal carina and rule out the chance of overly deep penetration of the endotracheal tube. The standard performance evaluation indicator includes but is not limited to precision, recall, mean average precision (mAP@50), and accuracy, to ensure the height accuracy of the model.

The distance zooming calculation entails performing ratio calculation on pixel values of the input image, predicted image, and training set image to convert the pixel values of the input image into the real distance by the predicted image and training set image, achieving precise image analysis and marking. The method is simple and easy enough to be widely applicable to image processing, enhancing the accuracy and efficiency of image analysis.

### Equipment Required for the Present Invention:

The use of the object detection module 2 and the position evaluation module 3 module does not require special equipment. Both of them are compatible with existing chest X-ray image collection equipment and medical systems and thus capable of offering a convenient integration solution. All clinical professionals or information systems need to do is input patients' X-ray image data to quickly attain an evaluation result of the endotracheal tube position.

In a preferred specific embodiment of the present invention, the chest X-ray image data of the object detection module 2 include a chest X-ray showing the presence of an endotracheal tube, a chest X-ray showing the presence of a tracheostomy tube, and a chest X-ray showing the absence of an endotracheal tube or tracheostomy tube.

In a preferred specific embodiment of the present invention, the chest X-rays of the endotracheal tube mainly originate from the chest X-rays of patients who need to undergo endotracheal tube-based therapy, including cases where the endotracheal tube positions are appropriate and cases where the endotracheal tube positions are inappropriate, with the patients differing in endotracheal tube experience and thoracic anatomical structures.

In a preferred specific embodiment of the present invention, the position evaluation module 3 triggers an alert when the endotracheal tube tip is lower than the tracheal carina or when the distance between the endotracheal tube tip and the tracheal carina is less than 3 cm or greater than 5 cm.

The embodiments below merely illustrate details and contents intended for comprehension of the present invention but not intended to limit the scope of the claims of the present invention.

In a preferred embodiment, the testing data originate from patients' chest X-ray images, including cases where the endotracheal tube positions are normal and cases where the endotracheal tube positions are incorrect, encompassing the diversity of different cases and different patients. The data is processed in a way to be rendered unidentifiable to protect patients' privacy and ensure compliance. The chest X-rays are taken of patients differing in endotracheal tube experience and thoracic anatomical structures.

Amount of data: 253 chest X-ray images are used in testing.

The testing result is as follows:
(1) sensitivity: 0.963
(2) specificity: 0.964
(3) mean average precision (mAP@50): 0.966
(4) accuracy: 0.962

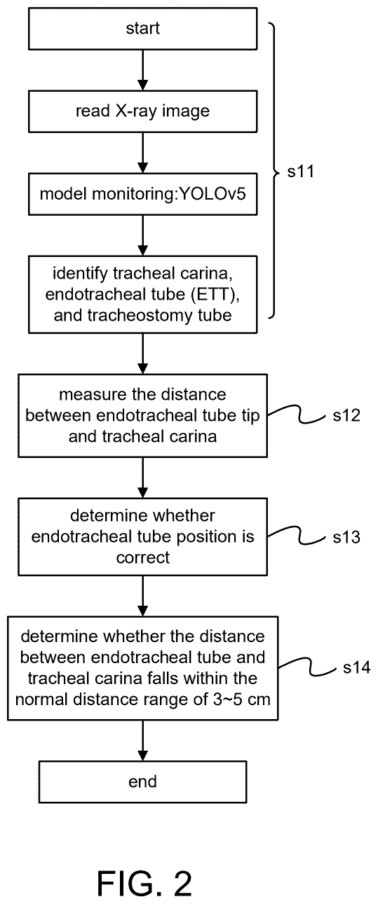

In a preferred embodiment, as shown in FIG. 2, its process flow is as follows:

Step s11 of model testing (YOLOv5): a pre-trained YOLOv5 model is used to test chest X-ray image data and identify the positions of the tracheal carina, endotracheal tube, and tracheostomy tube. As shown in FIG. 4B, the position of the tracheal carina is detected, but the endotracheal tube is absent from the image.

Step s12 of automatic identification of endotracheal tube tip position and automatic measurement of the distance between the endotracheal tube tip and the tracheal carina: the model automatically measures the distance between the endotracheal tube tip and the tracheal carina.

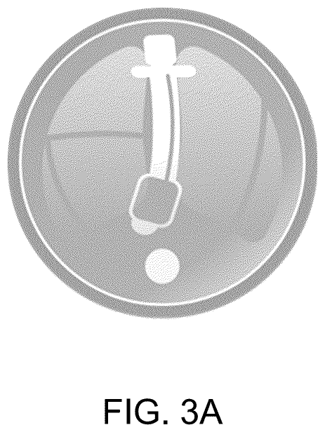

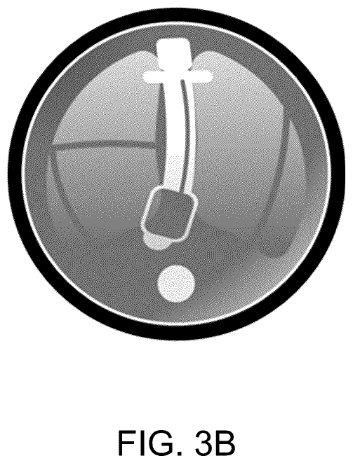

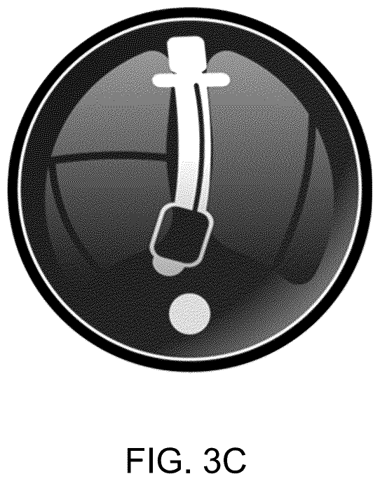

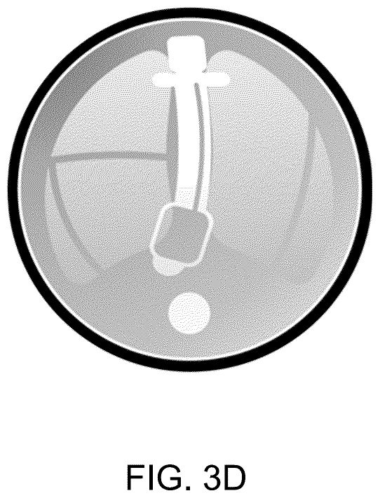

Step s13 of determining whether endotracheal tube position is correct: determining whether the position of the endotracheal tube is appropriate according to the endotracheal tube tip position and the measured distance between the endotracheal tube tip and the tracheal carina, as shown in FIG. 3B.

Step s14 of early alerting: sending an alert to warn of an incorrect endotracheal tube position whenever the endotracheal tube tip is lower than the tracheal carina or the measured distance does not lie in the range of 3˜5 cm, as shown in FIG. 3C.

FIG. 4 depicts the presence and positions of the tracheal carina, endotracheal tube, and tracheostomy tube shown in a chest X-ray image and identified by the artificial intelligence (AI) algorithm model and the measured distance between the endotracheal tube tip and the tracheal carina. Furthermore, consideration is given to past clinical standards and dissertations, as well as standard rules for endotracheal tube alert triggering, wherein the rules are as follows: display green light when the distance ranges from 3 to 5 cm, as shown in FIG. 3B; display yellow light when the distance ranges from 2.5 to 3 cm or from 5 to 5.5 cm, as shown in FIG. 3D; and display red light when the distance is less than 2.5 cm or greater than 5.5 cm, as shown in FIG. 3C. In another simplified embodiment, display red light when the distance is less than 3 cm or greater than 5 cm, as shown in FIG. 3C. The display occurs as shown in FIG. 3A when the tracheal carina is detected but the endotracheal tube is not detected. In a preferred embodiment, a new alert is strengthened by flash or sound, and the flash or sound pauses as soon as the clinical professionals become aware of the alert.

Therefore, a purpose of the present invention is based on the fact that sometimes accidental extubation or one lung ventilation (OLV) occurs to critically ill patients receiving mechanical ventilation, causing fatal injuries to the patients. Theoretically speaking, the position of an endotracheal tube shown in each chest X-ray image must be examined thoroughly. However, nowadays physicians are typically busy and thereby are focused on conspicuous lesions while reading chest X-rays; as a result, sometimes physicians are unaware of incorrect endotracheal tube positions. Therefore, the present invention provides an artificial intelligence (AI) algorithm model for assisting with automatic identification of the position of an endotracheal tube shown in a chest X-ray image. The applicable materials and method make it feasible to randomly choose critically ill patients' unidentifiable chest X-ray images to create a training dataset and a testing dataset, and a Python YOLOv5 model is used to create an artificial intelligence (AI) algorithm model, so as to predict the position of the tracheal carina shown in the chest X-ray image and confirm whether the endotracheal tube or tracheostomy tube is present. If the endotracheal tube is detected, it will be necessary to further calculate the distance between the tracheal carina and the endotracheal tube tip. If the endotracheal tube tip is below the tracheal carina or when the distance is less than 3 cm or greater than 5 cm, an alert will be triggered. Experimental results show that the present invention involves using 2278 chest X-ray images as a training dataset, and 253 chest X-ray images as a testing dataset, such that the created artificial intelligence (AI) algorithm model has precision, recall, mean average precision, and accuracy of 0.963, 0.964, 0.966 and 0.962 respectively. The present invention involves applying the artificial intelligence (AI) algorithm model to clinical practice to achieve external verification. Given an alerting system triggered by the artificial intelligence (AI) algorithm model, the median (interquartile range) of the duration of an inappropriate endotracheal tube position takes on a decreasing trend, decreasing from 3.00 (1.25˜4.00) days to 2.00 (1.00˜3.00) days. Therefore, the present invention provides an artificial intelligence (AI) algorithm model for assisting with automatically reading and determining the appropriateness of the position of the endotracheal tube shown in a chest X-ray image and sending an appropriate alert, with the artificial intelligence (AI) algorithm model exhibiting satisfactory performance during both the development stage and the testing stage and being applied to clinical practice to enhance patients' care quality and safety.

The present invention is applicable to the field of medical images, and its main purpose is to assist clinical professionals in quickly and accurately determining whether the position of the endotracheal tube is appropriate, so as to reduce unplanned endotracheal tube detachment rate or incidence rate of one lung ventilation (OLV) and thereby enhance the stability of vital signs of patients.

I. The present invention provides an endotracheal tube position anomaly alerting device that coordinates with an artificial intelligence (AI) algorithm module for evaluating endotracheal tube position appropriateness. The predetermined purposes or advantages of the artificial intelligence (AI) algorithm module are as follows:
1. Perform automatic evaluation on the position of an endotracheal tube shown in a patient's chest X-ray image.
2. Assist clinical professionals in quickly and accurately determining whether the position of an endotracheal tube is appropriate.
3. Reduce unplanned endotracheal tube detachment rate or incidence rate of one lung ventilation (OLV), reduce the risk of related complications, enhance medical care quality, and enhance patients' safety and stability of vital signs.

II. Indications for use of the endotracheal tube position anomaly alerting device of the present invention are as follows:
1. The indications of the artificial intelligence (AI) algorithm module include but are not limited to chest X-ray images taken of patients to determine whether the position of the endotracheal tube is appropriate.
2. The indications of the artificial intelligence (AI) algorithm module include all patients who need to undergo endotracheal tube-based therapy, including but not limited to critically ill patients and surgical patients.

Every patient staying in an intensive care unit (ICU) has to receive a chest X-ray every two to five days on average. Chest X-rays are nowadays read by physicians who are typically busy and thereby are focused on major lesions, such as pneumonia, while reading the chest X-rays; as a result, sometimes physicians are too busy to check the chest X-rays for the correctness of endotracheal tube positions one by one. Therefore, the endotracheal tube position anomaly alerting device of the present invention is effective in predicting the position of an endotracheal tube and monitoring, including selecting/detecting the position of the endotracheal tube to provide real-time evaluation results, serving as an auxiliary tool for providing endotracheal tube alerting to clinical professionals, so as to ensure an appropriate position of the endotracheal tube, enhance patients' therapeutical effects, and reduce related risks.

Therefore, the technical features of the present invention are as follows:
1. Performing model training with the YOLO V5 model, using 2278 chest X-ray image samples to perform training, and using 253 images to perform testing and verification.
2. X-ray Image Prediction:
   a. Predicting the position of the tracheal carina, and confirming whether an endotracheal tube or a tracheostomy tube is present;
   b. Further calculating the distance between the tracheal carina and the endotracheal tube tip and determining the appropriateness of the endotracheal tube position when the endotracheal tube is present.
3. Proof of Concept

Therefore, the present invention is an endotracheal tube position anomaly alerting device that is effective in overcoming the drawbacks of the prior art. The endotracheal tube position anomaly alerting device evaluates endotracheal tube position appropriateness, on the assumption that every patient staying in an intensive care unit (ICU) has to receive a chest X-ray every two to five days on average, by performing artificial intelligence (AI) algorithm model training with chest X-ray image samples and then using the artificial intelligence (AI) algorithm model to: 1. determine the position of a tracheal carina shown in a chest X-ray image, determine whether an endotracheal tube (ETT) or tracheostomy tube is in use, and determine its position; 2. calculate the appropriateness of the endotracheal tube position and send an alert. Therefore, the present invention is not only inventive and practical but also meets user needs.

The preferred embodiments herein disclosed are not intended to unnecessarily limit the scope of the invention. Therefore, simple modifications or variations belonging to the equivalent of the scope of the claims and the instructions disclosed herein for a patent are all within the scope of the present invention.

What is claimed is:

1. An endotracheal tube position anomaly alerting device, for monitoring correctness of a position of an endotracheal tube, comprising:
a monitoring module for collecting patients' chest X-ray image data;
an object detection module connected to the monitoring module to receive the chest X-ray image data from the monitoring module and use an artificial intelligence (AI) algorithm model to identify presence of a tracheal carina, an endotracheal tube (ETT) and a tracheostomy tube and mark positions thereof in the chest X-ray image data;
a position evaluation module connected to the object detection module to automatically measure a distance between an endotracheal tube tip and the tracheal carina upon detection of presence of the tracheal carina and the endotracheal tube by the object detection module and according to mark positions of a determination result of the artificial intelligence (AI) algorithm model, generate an evaluation result of the endotracheal tube position appropriateness according to the distance thus measured, and trigger an alert when the evaluation result indicates that the position of the endotracheal tube does not fall within a correct range; and
a display module connected to the position evaluation module to receive the determination result of the object detection module and the position evaluation module, send an alerting signal, and display an object marking result and the evaluation result of the endotracheal tube position appropriateness.

2. The endotracheal tube position anomaly alerting device according to claim 1, wherein the object detection module, based on a deep learning model of YOLOv5, inputs a plurality of chest X-ray image data, treats the plurality of chest X-ray image data as a training dataset and a testing dataset for deep learning according to a predetermined ratio, performs model training with the training dataset, performs testing with the testing dataset, performs verification to finish training the artificial intelligence (AI) algorithm model, and evaluates the performance of the artificial intelligence (AI) algorithm model according to a standard performance evaluation indicator.

3. The endotracheal tube position anomaly alerting device according to claim 2, wherein the standard performance evaluation indicator includes but is not limited to precision, recall, mean average precision (mAP@50), and accuracy.

4. The endotracheal tube position anomaly alerting device according to claim 1, wherein the position evaluation module measures the distance between the endotracheal tube tip and the tracheal carina by selecting the leftmost coordinate pair and the rightmost coordinate pair on the lower edge of a marking bounding box of the identified endotracheal tube tip and the midpoint between two points calculated with a function, calculating a distance from each of the three points to the tracheal carina with Euclidean distance equation, selecting the shortest distance, and converting pixel values of the distance in the image into the actual distance.

5. The endotracheal tube position anomaly alerting device according to claim 1, wherein the position evaluation module triggers an alert when the endotracheal tube tip is lower than the tracheal carina or when the distance between the endotracheal tube tip and the tracheal carina is less than 3 cm or greater than 5 cm.

6. The endotracheal tube position anomaly alerting device according to claim 1, wherein a means of triggering the alert includes but is not limited to a picture, a flashing picture or sound.

7. The endotracheal tube position anomaly alerting device according to claim 5, wherein a means of triggering the alert includes but is not limited to a picture, a flashing picture or sound.

8. The endotracheal tube position anomaly alerting device according to claim 1, wherein the chest X-ray image data of the object detection module includes chest X-ray images indicative of the presence of the endotracheal tube, chest X-ray images indicative of the presence of the tracheostomy tube, and chest X-ray images indicative of the absence of the endotracheal tube or tracheostomy tube.

9. The endotracheal tube position anomaly alerting device according to claim 8, wherein the chest X-ray images indicative of the presence of the endotracheal tube mainly originate from the chest X-rays of patients who need to undergo endotracheal tube-based therapy, including cases where the endotracheal tube positions are appropriate and cases where the endotracheal tube positions are inappropriate, with the patients differing in endotracheal tube experience and thoracic anatomical structures.
